(19) United States
(12) Patent Application Publication
(10) Pub. No.: US 2005/0109350 A1
(43) Pub. Date: May 26, 2005

(54) Method and system for biometric surgical confirmation

(76) Inventor: Michael Luloh, Chuluota, FL (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/737,609

(22) Filed: Dec. 16, 2003

Related U.S. Application Data
(60) Provisional application No. 60/524,834, filed on Nov. 25, 2003.

(51) Int. Cl. A61B 19/00

(52) U.S. Cl. 128/898

(57) ABSTRACT

A method and system for biometric confirmation of a patient with a surgery are disclosed. One embodiment of the method comprises: obtaining a first set of biometric data associated with the patient, during a first aspect of the surgery; associating the first set of biometric data with the surgery; obtaining a second set of biometric data associated with the patient, during a second aspect of the surgery; comparing the second set of biometric data to the first set of biometric data; and if the second set of biometric data and the first set of biometric data contain a match, then providing a match indication, else providing a mismatch indication. The first set of biometric data and the second set of biometric data each can comprise at least one biometric data type, wherein each biometric data type comprises one of fingerprint data, retinal scan data, iris/sclera scan data, eye blood vessel scan data, voice pattern data and DNA data. Further, the first set of biometric data and the second set of biometric data each should comprise at least one of the same biometric data type.

### TECHNICAL FIELD OF THE INVENTION

This application claims priority from U.S. Ser. No. 60/524,834, filed Nov. 25, 2003.

The present invention relates to the field of patient identification and, more particularly, to identification of human patients by biometric indicia. Even more particularly, the present invention relates to a method and system for biometric confirmation of a surgical procedure to a patient.

### BACKGROUND OF THE INVENTION

Ensuring that a patient receives the intended treatment for a medical condition is an ultimate goal of the medical profession. In particular, ensuring that a patient does not undergo an erroneous surgical procedure, or a correct surgical procedure on the wrong body part, is of paramount importance both for the health and safety of the patient and for liability concerns of the provider. It is well known that such errors sometimes occur. These situations are not only unfortunate, but can have disastrous and even deadly consequences for the patient. Further, the liability incurred by the surgical provider can be great. Although the exact number of such erroneous procedures is unknown, it is an ultimate goal to reduce and preferably eliminate the opportunity for error when it comes to matching a patient with an intended surgical procedure. In such cases, even a single error is undesirable.

The number and complexity of surgical procedures is increasing every day, with a resultant increase in the likelihood that an error, such as described above, will occur. Currently, most such errors are avoided by conscientious and rigid requirements on surgical personnel to crosscheck the identity of a patient against the intended surgical procedure. However, even the most rigid requirements on personnel cannot eliminate the likelihood of human error, especially when dealing with a large number of patients in a rapidly moving and hurried clinical environment. Further, cross-checking a patient to the intended surgical procedure typically consists of checking a patient's identification bracelet to a chart. Under such circumstances, the possibility exists that a patient is assigned a wrongly labeled bracelet, or that the patient is correctly matched to a procedure, but the procedure is performed on the wrong body part. Positively identifying a patient and matching the patient to the correct surgical procedure, on the correct body part, is thus becoming increasingly important. Current methods for confirming a patient-surgery match are not sufficiently reliable, or even cost effective, in today's surgical climate.

Biometric indicia are used for identification purposes in many sectors. For example, perhaps the most familiar example of biometric indicia are fingerprints, which are commonly used for identification of personnel for security purposes, and for identifying suspects in criminal investigations. Other biometric identification systems include inkless fingerprint systems (called “live scan” units), retinal scanners, hand geometry measuring devices, voice recognition, handwriting recognition, and facial recognition systems that use either visual or infrared cameras. Because they are reliable, biometric identification systems of these types are typically used for access control and for tracking or identifying persons following some event. It is not known, however, to use biometric indicia to correlate a patient with a surgical procedure to ensure that the patient does not receive erroneous treatment.

Therefore, a need exists for a method and system for biometric surgical confirmation that can reduce or eliminate the problems associated with previous methods for matching a patient to a surgical procedure in a surgical environment. The benefits of a method and system for biometric surgical confirmation are obvious and great, in that the undesirable consequences of unnecessary and/or unwanted surgeries, including damage to a patient's health and the related costs, are reduced or eliminated.

### BRIEF SUMMARY OF THE INVENTION

The embodiments of the method and system for biometric surgical confirmation of the present invention substantially meet these needs and others. One embodiment of the method for biometric confirmation of a patient with a surgery comprises: obtaining a first set of biometric data associated with the patient, during a first aspect of the surgery; associating the first set of biometric data with the surgery; obtaining a second set of biometric data associated with the patient, during a second aspect of the surgery; comparing the second set of biometric data to the first set of biometric data; and if the second set of biometric data and the first set of biometric data contain a match, then providing a match indication, else providing a mismatch indication.

The first set of biometric data and the second set of biometric data each can comprise at least one biometric data type, wherein each biometric data type comprises one of fingerprint data, retinal scan data, iris/sclera scan data, eye blood vessel scan data, voice pattern data and DNA data. Further, the first set of biometric data and the second set of biometric data each should comprise at least one of the same biometric data type. It should be noted that performing the surgery comprises performing a final aspect of the surgery, where the final aspect of the surgery involves surgically altering a physical aspect of the patient. The first and the second aspects of the surgery are preparatory steps to actually performing a surgical alteration on the patient.

Further embodiments to the present invention can include a system for biometric confirmation of a patient with a surgery in accordance with the teachings of this invention. One embodiment of the system can comprise: a first biometric input device operable to obtain a first and a second set of biometric data associated with the patient; means for associating the first set of biometric data with the surgery; means for comparing the first set of biometric data with the second set of biometric data to identify if a match exists; and means for providing a match indication if a match exists and for providing a mismatch indication if a match does not exist. The system can further comprise a second biometric input device operable to obtain the second set of biometric data. In this case, the first biometric input device obtains the first set of biometric data

The first biometric input device can further comprise a diagnostic console for receiving the first and the second sets of biometric data and means for storing the first set of biometric data. Alternatively, the second biometric input device can be communicatively connected to a surgical console. The means for storing the first set of biometric data can comprise a floppy disk, a compact disk, a barcode, or any other data storage means as known to those in the art.

Embodiments of the method and system of this invention can be implemented within any surgical environment, and in particular can be implemented within a refractive surgery environment. For example, embodiments of this invention can be implemented within the LadarVision® System manufactured and sold by Alcon Laboratories, Inc. of Fort Worth, Tex. Thus, embodiments of this invention can be incorporated within a surgical machine or system for use in ophthalmic, refractive or other surgery. Other uses for a method and/or system for biometric surgical confirmation designed in accordance with the teachings of this invention will be known to those familiar with the art and are contemplated to be within the spirit and scope of the present invention.

### BRIEF DESCRIPTION OF THE SEVERAL VIEWS OF THE DRAWINGS

A more complete understanding of the present invention and the advantages thereof may be acquired by referring to the following description, taken in conjunction with the accompanying drawings, in which with like reference numbers indicate like features and wherein:

FIG. 1 is a simplified block diagram of a surgical system 10 comprising one or more biometric input devices in accordance with the teachings of this invention; and

FIG. 2 is a simplified flow chart illustrating one embodiment of the method for biometric confirmation of the present invention.

### DETAILED DESCRIPTION OF THE INVENTION

Preferred embodiments of the present invention are illustrated in the FIGUREs, like numerals being used to refer to like and corresponding parts of the various drawings.

The various embodiments of the present invention provide a method and system for biometric confirmation for use with surgical procedures, such as refractive laser eye surgery. Embodiments of the system of this invention can comprise, for example, a surgical system (e.g., a refractive eye surgery system), such as known to those in the art, coupled with a biometric measurement device for detecting, measuring and storing biometric indicia of a patient and associating the patient's biometric indicia with a desired surgical procedure. The surgical system or device can be a single piece, or a multiple piece, system, wherein the biometric patient information can be acquired as a first step of a surgical procedure at a first unit of the surgical system, and wherein the surgery itself can be performed at a second unit of the surgical system wherein the first and second units can be one and the same, or separate units.

For example, patient biometric information can first be taken as part of a diagnostic step, such as during a wavefront analysis for refractive surgery, or as a stand alone step of taking and associating biometric data with a surgical procedure. The same biometric information can then be taken a second time for confirmation against the first set of biometric information. The second set of biometric patient information can be taken at a second unit of the surgical system, such as at a refractive laser console, where the refractive surgery is actually performed.

Embodiments of the method and system for biometric surgical confirmation of the present invention are discussed herein with regards to use in the general field of ophthalmic surgery, and in particular, refractive laser surgery. However, it is contemplated and it will be realized by those skilled in the art that the scope of the present invention is not limited to ophthalmology, but may be applied generally to other areas of surgery where biometric confirmation of a patient against a surgery may be required or desired.

An embodiment of the system for biometric surgical confirmation of this invention can comprise a biometric measurement device, such as a fingerprint sensor, operably connected to a diagnostic unit and/or a surgical unit of a surgical system. Alternatively, the biometric device can be a stand-alone device. For example, a biometric measuring device can be incorporated into a wavefront measuring device of a refractive surgical system and/or into the surgical laser component of the refractive surgical system. When taking diagnostic information, such as wavefront measurements, from a patient, the patient can be prompted, for example, to place a thumb on a fingerprint sensor to acquire the patient's unique fingerprint data. The patient's biometric (e.g., fingerprint) data is associated with the patient's intended surgery to ensure that only the intended surgery is performed on the patient. The patient's fingerprint data can then be transferred, along with the diagnostic information, to the surgical console of the surgical system.

Prior to performing the surgery, the surgeon, or another member of the surgical team, can prompt the patient to again place the same finger (thumb) on a fingerprint sensor at the surgical unit, resulting in a repeated acquisition of the patient's fingerprint data. This second set of fingerprint data is compared, for example, in software, to the first set of patient fingerprint data. Only if the two sets of patient data match will the surgical unit allow the surgeon to proceed with the surgery. Otherwise, the surgical unit will prevent the surgery from proceeding until a matching set of biometric patient data is provided, unless a member of the surgical staff manually overrides the surgical unit. A manual override should only occur after extreme caution has been applied to ensure that the patient, body part, and surgery properly correlate.

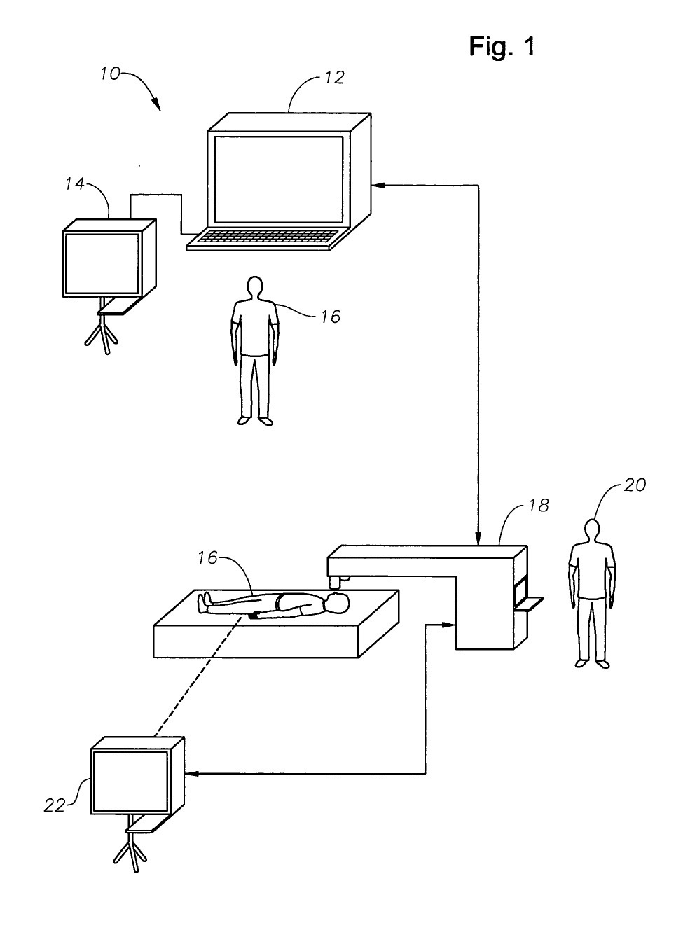

FIG. 1 is a simplified block diagram of a surgical system 10 comprising one or more biometric input devices 14 and 22 in accordance with the teachings of this invention. Surgical system 10 further comprises a diagnostic console 12 operably coupled to diagnostic biometric input device 14. Diagnostic biometric input device 14 can be used for taking a first set of biometric data associated with a patient 16. The first set of biometric data can be taken, for example, during a diagnostic procedure associated with a contemplated surgery. The diagnostic procedure can comprise, for example, a wavefront analysis for a refractive laser procedure, or can be as simple as taking blood pressure, body temperature and/or any other such commonly measured patient parameter. The biometric data measured by diagnostic biometric input device 14 can comprise one or more biometric data types, such as fingerprint data, retinal scan, iris/sclera scan, eye blood vessel scan, voice pattern, DNA or any other such biometric data types as are known to those familiar with the art.

Diagnostic biometric input device 14 can provide the first set of patient biometric data to diagnostic console 12, which associates the first set of patient biometric data with an intended surgical procedure based on, for example, a doctor's entry and patient confirmation of the surgery at the time of providing the biometric data. Diagnostic console 12 can forward the first set of patient biometric data and associated surgery data to a surgical console 18, which is communicatively connected to diagnostic console 12. Alternatively, diagnostic biometric input device 14 can forward the first set of patient biometric data directly to a surgical console 18, or the first set of patient biometric data can be stored on a removable media for manual transport to the surgical console 18 or other device.

Following the acquisition of the first set of patient biometric data, the first set of patient biometric data can be stored until the time at which the subsequent surgical procedure is to take place. Note that the time between a diagnostic aspect (or biometric data entry aspect) and the surgical aspect of a surgical procedure can vary, such that the surgical aspect of the surgical procedure can immediately follow the diagnostic aspect of the surgical procedure, or it can occur at some later time separated by hours, days, or any such time period. Once patient 16 is ready to undergo the surgical procedure, patient 16 is situated, as appropriate, on or near surgical console 18, depending on the surgical procedure to be performed. Before the surgery can begin, the surgeon or other surgical team member 20 will prompt patient 16 to provide, via surgical biometric input device 22, a second set of the same biometric data.

Surgical biometric input device 22 measures the biometric data from patient 16 and provides it to surgical console 18, to which surgical biometric input device 22 is communicatively connected. Surgical console 18 can compare the first set of patient biometric data to the second set of patient biometric data measured by surgical biometric input device 22. The comparison can be performed by various means, as known to those familiar with the art. For example, the surgeon, or other personnel, can physically compare the two sets of biometric data, or the comparison can be performed electronically by means of software.

If the two sets of patient biometric data match, then surgical console 18 allows the surgery to proceed. If the two sets of patient biometric data do not match, surgical console 18 will lock itself down and prevent the surgeon 20 from performing the surgery until matching patient biometric data is provided. Alternatively, it is contemplated that a manual override can be incorporated within surgical console 18 such that a surgeon or other surgical team member 20 can override surgical console 18 and allow the surgical procedure to proceed even when the sets of biometric patient data do not match. It is contemplated that such a surgical team override will only occur after extreme caution has been taken to ensure that the intended patient, body part and surgery have been correlated.

Surgical system 10 can comprise discrete and separate diagnostic and surgical consoles 12 and 18, or diagnostic and surgical functions can be incorporated within a single console. In such a case, a single biometric input device 14 or 18 can be used for measuring and storing both the first set of patient biometric data and the second set of patient biometric data. In such an embodiment, matching of the two sets of patient biometric data and the surgical lockout discussed above will still function in the manner disclosed.

The embodiments of the method and system for biometric surgical confirmation of the present invention can thus be used to effectively prevent a surgical procedure from being performed on either the wrong patient or on the wrong body part of a correct patient. In particular, with regards to refractive laser eye surgery, experience has shown that occasionally a surgical staff member may match the wrong patient, and correspondingly the wrong patient parameters, to a surgical procedure. This is because, typically, when a staff member of a refractive surgery clinic prepares a refractive laser eye surgery device to perform surgery on a patient, the staff member must select the appropriate patient from a long list of displayed patients on a display device, such as a computer monitor. It is thus possible to mistakenly select from this list a patient other than the patient currently awaiting surgery. Further, experience has shown that occasionally a member of the surgical staff will select the wrong eye of the correct patient. Such mistakes are likely to result in injury to the patient and in other complications. The method and system for biometric surgical confirmation of the present invention will greatly reduce or eliminate such erroneous patient-surgery correlations.

In some embodiments of this invention, the first set of biometric data can be stored or encoded in a tangible medium using a storage means such as a floppy disk, a compact disk (“CD”), or other magnetic or optical storage media, or on a barcode or similar physical media. The first set of biometric data can then be physically associated with a patient (e.g., attached to a patient's surgical chart). The first set of biometric data can thus be physically carried to a surgical console, or other location, and scanned in to a receiving device (e.g., the surgical console) via, for example, a barcode scanner, floppy drive, or other corresponding device. The first set of biometric data can then be compared to a second set of patient biometric data as previously described herein. Note that the second set of biometric data can likewise be stored or encoded in a tangible medium. Further, it is contemplated that the first and second set of patient biometric data can comprise a single type of biometric data (e.g., fingerprints) or a plurality of biometric data types in any combination (e.g., fingerprints, retinal scans, iris scans, etc.). In such embodiments, diagnostic biometric input device 14 and surgical biometric input device 22 will comprise multiple input sensors as appropriate for the types of biometric data they will measure. Further, in such embodiments at least one type of biometric data in each of the first and second sets of biometric data must be the same to perform matching of the patient's biometric data.

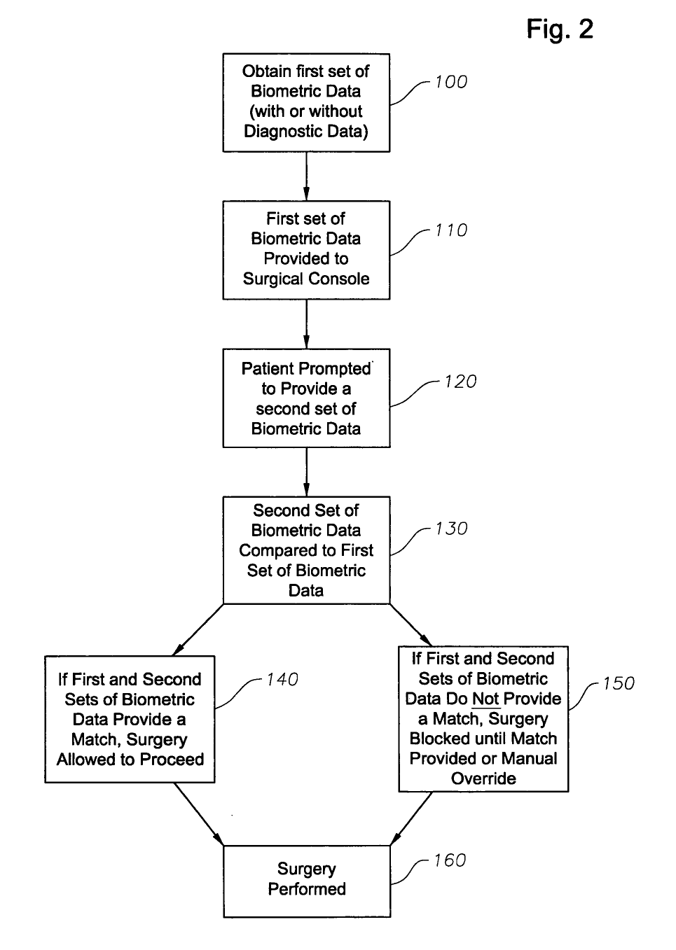

FIG. 2 is a simplified flow chart illustrating one embodiment of the method for biometric surgical confirmation of the present invention. At Step 100, diagnostic information associated with a patient 16 is taken during a diagnostic aspect of a contemplated surgical procedure. This diagnostic information can comprise, for example, a wavefront analysis of a patient's cornea in anticipation of refractive laser surgery, or any other diagnostic information as known to those familiar with the art (e.g., blood pressure, body temperature). During the diagnostic aspect at Step 100, a first set of biometric data associated with the patient 16 is taken using a biometric input device, such as diagnostic or surgical biometric input devices 14 and 22 of FIG. 1. Alternatively, at step 100, only biometric data can be measured and recorded, along with a patient identifier, such as the patient's name. At Step 110, the diagnostic information and/or the first set of patient biometric data is provided to a surgical device, such as surgical console 18. The diagnostic information and/or biometric data can be provided to surgical console 18 either by an electronic communications link, such as a direct computer-to-computer link, wired or wireless network link, internet link, or other such electronic connection known to those in the art. Alternatively, the data can be physically transferred to surgical console 18 in the form of a barcode or other physical media, or as electronically encoded information, for example, on a floppy disk, CD or other optical or magnetic media.

At Step 120, once a patient is prepared and ready for surgery a surgical team member prompts the patient to re-enter one or more of the same type of biometric data as comprises the first set of biometric data, at a surgical biometric input device, such as surgical biometric input device 22 of FIG. 1. It is contemplated that the surgical team member can be prompted by surgical console 18 to prompt the patient 16 to re-enter biometric data before surgery is allowed to be performed. At Step 130, the second set of biometric data is compared to the first set of biometric data at, for example, surgical console 18 or diagnostic console 12, as it is contemplated that diagnostic console 12 and surgical console 18 can be in electronic communication with one another, or at surgical console 18 if the first set of biometric data was provided in some physical media to surgical console 18.

At Step 140, if the first and second sets of patient biometric data match, a match indication is provided to, for example, the surgeon or surgical team, and the surgical procedure is allowed to go forth at step 160. If instead the two sets of patient biometric data do not match, then at step 150 the surgical console 18 will automatically block the surgeon and the surgical team from performing the surgical procedure. The block can comprise an indication to the surgeon or other personnel that the two sets of biometric data do not include a match. The block will remain in place until a matching set of biometric data is entered into surgical console 18 via surgical biometric input device 22 (e.g., the correct patient is identified and prepped for the surgery, or an erroneous reading is corrected by, for example, taking a second set of biometric data from the same patient).

Alternatively, it is contemplated that surgical console 18 can include a manual override function for the non-matching biometric data block, wherein a surgeon or other surgical team member 20 can override the surgical block following a careful review to ensure that the proper patient, body part and surgical procedure correlation exists. This embodiment of the method of this invention then proceeds to Step 160, where the surgery is allowed to proceed following either a match of the two sets of patient biometric data at Step 140, or a manual override at Step 150. At Step 160 the final aspect of the contemplated surgical procedure (e.g., the surgical alteration of a physical aspect of the patient) is performed.

An advantage of the embodiments of the method and system for biometric surgical confirmation of the present invention is that they will significantly reduce or eliminate the occurrence of patient-surgery mismatches. The method and system of this invention will provide both the patient and the surgical staff an added level of assurance and comfort knowing that, as much as possible, the patient, body part, and surgery have been properly correlated. Further, the method and system of this invention have the advantage of a very visible, to the patient, system for ensuring that erroneous surgical procedures will not be performed on the patient. The likelihood that fearful patients will undertake necessary surgeries can thus be increased.

Although the present invention has been described in detail herein with reference to the illustrated embodiments, it should be understood that the description is by way of example only and is not to be construed in a limiting sense. It is to be further understood therefore that numerous changes in the details of the embodiments of this invention and additional embodiments of this invention will be apparent to, and may be made by, persons of ordinary skill in the art having reference to this description. It is contemplated that all such changes and additional embodiments are within the spirit and true scope of this invention as claimed below. Thus, while the present invention has been described with reference to the general area of ophthalmic surgery, and, in particular, with reference to refractive laser surgery, the teachings contained herein apply equally wherever it is desirous to provide proper patient and/or body part and surgical procedure correlation.

1. A method for biometric confirmation of a patient with a surgery, comprising:
obtaining a first set of biometric data associated with the patient, during a first aspect of the surgery;
associating the first set of biometric data with the surgery;
obtaining a second set of biometric data associated with the patient, during a second aspect of the surgery;
comparing the second set of biometric data to the first set of biometric data; and
if the second set of biometric data and the first set of biometric data contain a match, then providing a match indication, else providing a mismatch indication.

2. The method of claim 1, wherein the first set of biometric data and the second set of biometric data each comprise at least one biometric data type, wherein each biometric data type comprises one of fingerprint data, retinal scan data, iris/sclera scan data, eye blood vessel scan data, voice pattern data and DNA data.

3. The method of claim 2, wherein the first set of biometric data and the second set of biometric data each comprise at least one of the same biometric data type.

4. The method of claim 3, wherein a match between the first set of biometric data and the second set of biometric data comprises matching data between at least one data type.

5. The method of claim 2, wherein obtaining the first set of biometric data and obtaining the second set of biometric data each comprise obtaining data from the patient in the form of one or more of the biometric data types and storing the biometric data.

6. The method of claim 5, wherein the first set of biometric data is obtained at a first biometric input device and the second set of biometric data is obtained at a second biometric input device, and wherein the first and the second biometric input devices are operable to receive the corresponding set of biometric data in the form of at least one of the biometric data types.

7. The method of claim 6, wherein, wherein the first biometric input device and the second biometric input device are the same device.

8. The method of claim 1, wherein the first aspect of the surgery is a diagnostic aspect for taking patient diagnostic information.

9. The method of claim 1, wherein the first aspect of the surgery comprises taking identifying patient information, wherein the identifying patient information comprises at least the first set of biometric data and a patient identifier, wherein the patient identifier comprises a patient name.

10. The method of claim 1, wherein the second aspect of the surgery comprises a pre-surgery patient preparation step.

11. The method of claim 1, wherein associating the first set of biometric data with the surgery comprises physically or electronically linking the first set of biometric data with the surgery such that the second set of biometric data can be compared to the first set of biometric data before the surgery can be performed.

12. The method of claim 1, wherein the match indication comprises an approval for performing the surgery on the patient.

13. The method of claim 1, wherein the mismatch indication comprises a block on performing the surgery on the patient.

14. The method of claim 13, wherein the block comprises an electronic block on a surgical console for performing the surgery.

15. The method of claim 14, wherein the electronic block can be overridden and the surgery allowed to be performed even if a match between the first and the second sets of biometric data does not exist.

16. The method of claim 1, further comprising the step of, if the second set of biometric data and the first set of biometric data do not contain a match, overriding the block and allowing the surgery to be performed.

17. The method of claim 1, wherein performing the surgery comprises performing a final aspect of the surgery, wherein the final aspect of the surgery comprises surgically altering a physical aspect of the patient.

18. The method of claim 1, wherein the surgery comprises a refractive laser eye surgery.

19. A system for biometric confirmation of a patient with a surgery, comprising:
a first biometric input device operable to obtain a first and a second set of biometric data associated with the patient;
means for associating the first set of biometric data with the surgery;
means for comparing the first set of biometric data with the second set of biometric data to identify if a match exists; and
means for providing a match indication if a match exists and for providing a mismatch indication if a match does not exist.

20. The system of claim 19, wherein the first biometric input device obtains the first set of biometric data, and further comprising a second biometric input device operable to obtain the second set of biometric data.

21. The system of claim 20, wherein the first biometric input device comprises a diagnostic console and means for storing the first set of biometric data.

22. The system of claim 20, wherein the second biometric input device is communicatively connected to a surgical console.

23. The system of claim 19, further comprising means for storing the first set of biometric data.

24. The system of claim 23, wherein the means for storing comprise one of a floppy disk, a compact disk, and a barcode.

25. The system of claim 19, further comprising a surgical console communicatively connected to the first biometric input device for receiving the first and the second sets of biometric data.

26. The system of claim 25, further comprising means for blocking performing the surgery if a match does not exist and means for overriding the block.

27. The system of claim 19, wherein the first set of biometric data and the second set of biometric data each comprise at least one biometric data type, wherein each biometric data type comprises one of fingerprint data, retinal scan data, iris/sclera scan data, eye blood vessel scan data, voice pattern data and DNA data.

28. The system of claim 27, wherein the first set of biometric data and the second set of biometric data each comprise at least one of the same biometric data type.

29. The system of claim 28, wherein a match between the first set of biometric data and the second set of biometric data comprises matching data between at least one data type.

30. The system of claim 27, wherein obtaining the first set of biometric data and obtaining the second set of biometric data each comprise obtaining data from the patient in the form of one or more of the biometric data types and storing the biometric data.

31. The system of claim 30, wherein the first set of biometric data is obtained at the first biometric input device and the second set of biometric data is obtained at a second biometric input device, and wherein the first and the second biometric input devices are operable to receive the corresponding set of biometric data in the form of at least one of the biometric data types.

32. The system of claim 19, wherein associating the first set of biometric data with the surgery comprises physically or electronically linking the first set of biometric data with the surgery such that the second set of biometric data can be compared to the first set of biometric data before the surgery can be performed.

33. The system of claim 19, wherein the match indication comprises an approval for performing the surgery on the patient.

34. The system of claim 19, wherein the mismatch indication comprises a block on performing the surgery on the patient.

35. The system of claim 34, wherein the block comprises an electronic block on a surgical console operable to perform the surgery.

36. The system of claim 35, wherein the electronic block can be overridden and the surgery allowed to be performed even if a match between the first and the second sets of biometric data does not exist.

37. The system of claim 19, wherein the surgery comprises a refractive laser eye surgery.

38. The system of claim 19, wherein the means for associating, the means for comparing and the means for providing each comprise software operable to associate, compare and provide, respectively.
